(19) United States
(12) Patent Application Publication
(10) Pub. No.: US 2022/0054292 A1
(43) Pub. Date: Feb. 24, 2022

(54) Grip Support Orthosis

(71) Applicant: Robert Clay Taylor, Franklin, NC (US)

(72) Inventor: Robert Clay Taylor, Franklin, NC (US)

(21) Appl. No.: 16/997,172

(22) Filed: Aug. 19, 2020

(51) Int. Cl. A61F 5/01; A44B 18/00

(52) U.S. Cl. CPC A61F 5/0118; A44B 18/0069

(57) ABSTRACT

A grip support orthosis includes a brace configured to be worn by a user such that a wrist of the user is positioned in the brace at a wrist end portion of the brace and a forearm of the user is positioned in the brace at a forearm end portion of the brace. A plurality of mounting straps is configured for selectively securing the brace on the user. A first end portion of an elongated strap is fixed to the brace. A second end portion of the elongated strap is selectively mountable to the brace. A length of the elongated strap is no less than ten inches. The elongated strap is wrappable onto an object in order to assist the user with gripping the object.

### FIELD OF THE INVENTION

The present subject matter relates generally to an orthosis for assisting a wearer with gripping an object.

### BACKGROUND OF THE INVENTION

Orthoses are frequently worn to assist a wearer. For example, orthoses can support a body segment, assist movement, aid rehabilitation, etc. As a particular example, an arm or wrist orthosis can assist an individual with a paralyzed hand or arm. However, known arm or wrist orthoses suffer various drawbacks. An improved orthosis for aiding individuals with a paralyzed hand or arm would be welcome in the art.

### BRIEF DESCRIPTION OF THE INVENTION

Aspects and advantages of the invention will be set forth in part in the following description, or may be apparent from the description, or may be learned through practice of the invention.

In an example embodiment, a grip support orthosis includes a brace having a wrist end portion and a forearm end portion. The brace is configured to be worn by a user such that a wrist of the user is positioned in the brace at the wrist end portion of the brace and a forearm of the user is positioned in the brace at the forearm end portion of the brace. The grip support orthosis also includes a plurality of mounting straps on the brace. The plurality of mounting straps is configured for selectively securing the brace on the user. An elongated strap extends between a first end portion and a second end portion. The first end portion of the elongated strap is fixed to the brace, e.g., at the wrist end portion of the brace. The second end portion of the elongated strap is selectively mountable to the brace. The elongated strap has a length between the first and second end portions of the elongated strap. The length of the elongated strap is no less than ten inches (10″). The elongated strap is wrappable onto an object between the first and second end portions of the elongated strap in order to assist the user with gripping the object.

In a first example aspect, the length of the elongated strap is no less than twelve inches (12″) and no greater than thirty-six inches (36″).

In a second example aspect, the plurality of mounting straps includes a plurality of hook-and-loop mounting straps.

In a third example aspect, the plurality of mounting straps includes no less than three (3) mounting straps.

In a fourth example aspect, a support bar is disposed within the brace. The support bar is positioned proximate a bottom side of the brace.

In a fifth example aspect, the support bar extends between the wrist end portion and the forearm end portion of the brace within the brace.

In a sixth example aspect, the support bar is bent at the wrist end portion of the brace in order to accommodate the wrist of the user when the brace is worn by the user.

In a seventh example aspect, the support bar is an aluminum support bar.

In an eighth example aspect, the brace defines a slot at a top side of the brace. The slot extends between the wrist end portion and the forearm end portion of the brace. The plurality of mounting straps extends across the slot.

In a ninth example aspect, the bottom side of the brace is positioned on an underside of the forearm of the user when the brace is worn by the user, and the top side of the brace is positioned on a topside of the forearm of the user when the brace is worn by the user.

In a tenth example aspect, the brace defines an opening at the wrist end portion of the brace. The opening is configured for receipt of a thumb of the user.

In an eleventh example aspect, the second end portion of the elongated strap includes one or both of hooks and loops of a hook-and-loop fastener interface for selectively mounting the second end portion of the elongated strap to the brace.

In a twelfth example aspect, the brace is a leather brace.

Each of the example aspects recited above may be combined with one or more of the other example aspects recited above in certain embodiments. For instance, all of the twelve example aspects recited above may be combined with one another in some embodiments. As another example, any combination of two, three, four, five, or more of the twelve example aspects recited above may be combined in other embodiments. Thus, the example aspects recited above may be utilized in combination with one another in some example embodiments. Alternatively, the example aspects recited above may be individually implemented in other example embodiments. Accordingly, it will be understood that various example embodiments may be realized utilizing the example aspects recited above.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures.

FIG. 1 is a side, elevation view of a grip support orthotic according to an example embodiment of the present subject matter.

FIG. 2 is another side, elevation view of the example grip support orthotic of FIG. 1.

FIG. 3 is a top, plan view of the example grip support orthotic of FIG. 1.

FIG. 4 is a perspective view of the example grip support orthotic of FIG. 1 secured to a user.

FIG. 5 is another perspective view of the example grip support orthotic of FIG. 4 with an elongated strap of the example grip support orthotic wound onto a bar.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

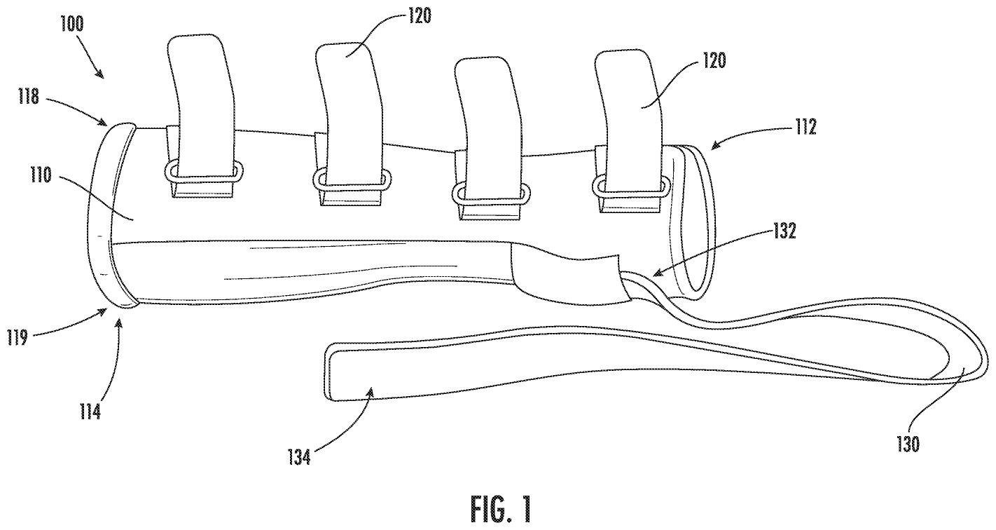

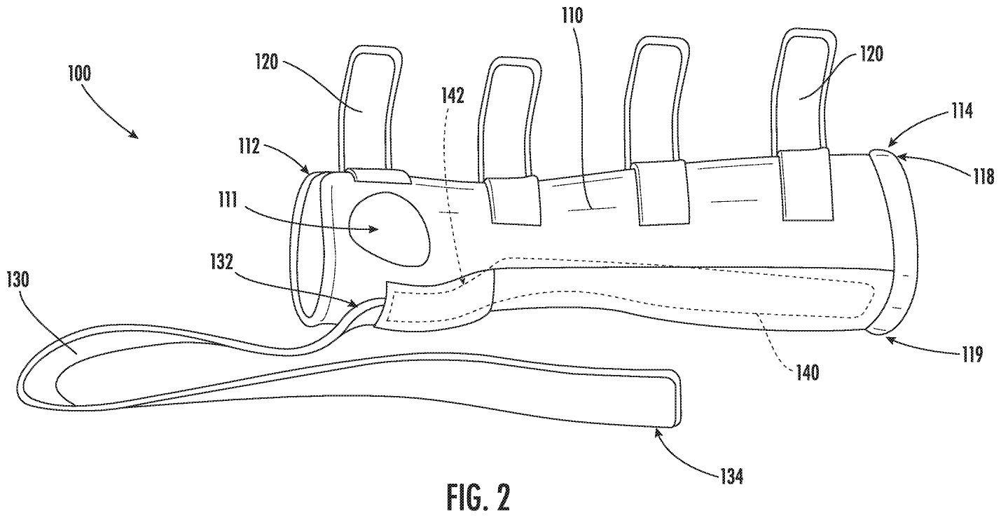

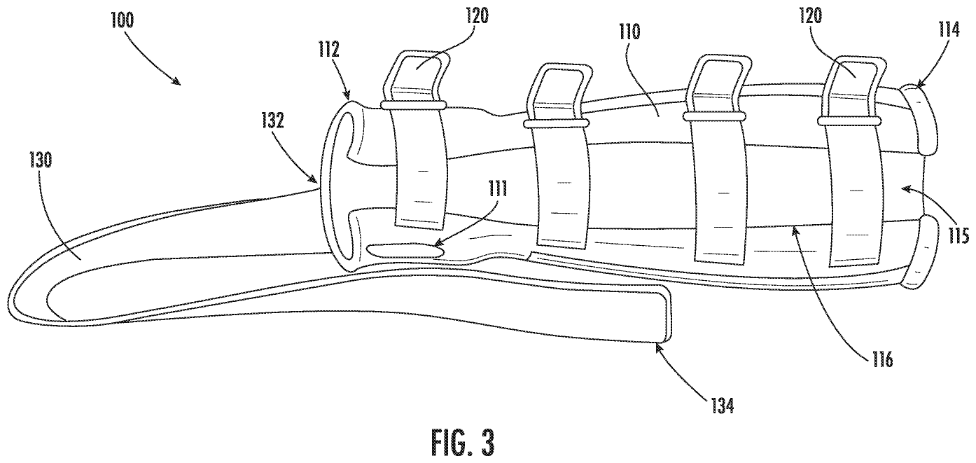

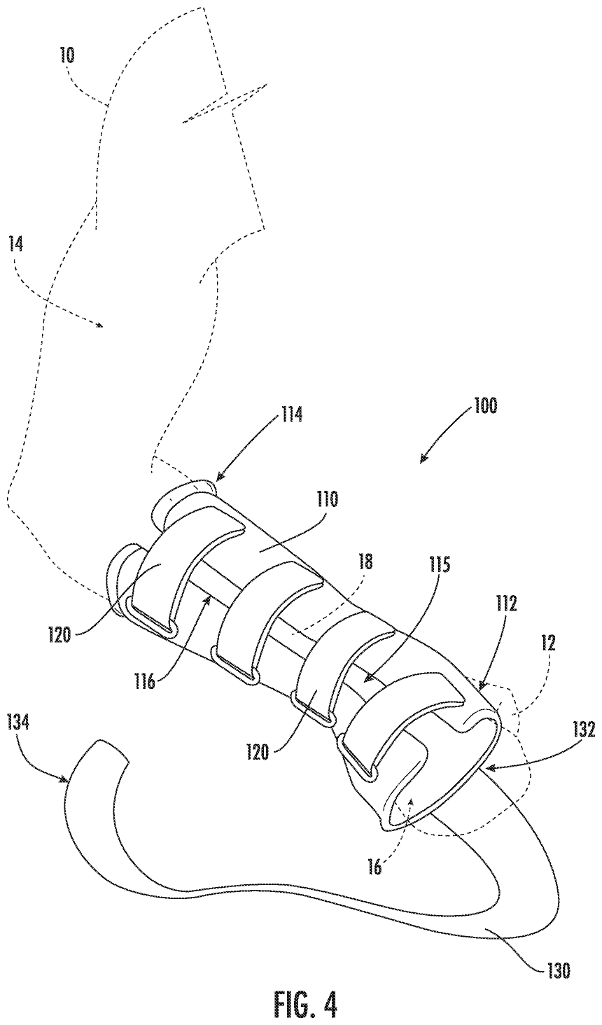

FIGS. 1 and 2 are side, elevation views of a grip support orthotic 100 according to an example embodiment of the present subject matter. FIG. 3 is a top, plan view of grip support orthotic 100, and FIG. 4 is a perspective view of grip support orthotic 100 secured to a user 10. As discussed in greater detail below, grip support orthotic 100 includes features for assisting user 10 with gripping an object. In particular, a hand 12 and/or arm 14 of user 10 may be, e.g., partially, paralyzed. Utilizing grip support orthotic 100, user 10 may engage in a wide variety of activities that build strength in hand 12 and/or arm 14 of user 10. For instance, grip support orthotic 100 may assist user 10 during weightlifting, yard work, physical therapy, etc.

With reference to FIGS. 1 through 4, grip support orthotic 100 may include a brace 110, a plurality of mounting straps 120, and an elongated strap 130. Brace 110 is configured to worn by user 10. In particular, brace 110 may have a wrist end portion 112 and a forearm end portion 114, and brace 110 may be tubular and/or hollow such that at least a portion of arm 14 of user is receivable within brace 110. For instance, brace 110 may be configured to be worn by user 10 such that a wrist 16 of user 10 is positioned in brace 110 at wrist end portion 112 of brace 110 and a forearm 18 of user 10 is positioned in brace 110 at forearm end portion 14 of brace 110. Brace 110 may be constructed of any suitable material. For instance, brace 100 may be constructed of leather, fabric, etc.

As may be seen from the above, an interior 115 brace 110 may accessible to allow user 10 to insert wrist 16 and/or forearm 18 of user 10 into interior 115 of brace 110. For example, user 10 may insert wrist 16 into interior 115 of brace 110 at forearm end portion 14 of brace 110, and user 10 may slide wrist 16 through interior 115 of brace 110 until wrist 16 of user 10 is positioned in brace 110 at wrist end portion 112 of brace 110 and forearm 18 of user 10 is positioned in brace 110 at forearm end portion 14 of brace 110. Thus, interior 115 of brace 110 may be open at wrist end portion 112 and forearm end portion 114 of brace 100. As another example, brace 110 may define a slot 116 at a top side 118 of brace 110. Slot 116 may extend between wrist end portion 112 and forearm end portion 114 of brace 100. User 110 may insert wrist 16 and forearm 18 of user 10 through slot 116 into interior 115 of brace 110. Thus, interior 115 of brace 110 may be open at slot 116, and slot 116 may assist user 10 with quickly mounting brace 110 on wrist 16 and forearm 18. Brace may also define an opening 111 at wrist end portion 112 of brace 110. Opening 111 may be configured for receipt of a thumb of user 10. Opening 111 may facilitate a comfort of user wearing brace 110.

Mounting straps 120 may be positioned on brace 110 and may be configured for selectively securing brace 110 on user 10. For instance, mounting straps 120 may extend across slot 116. User 10 may cinch mounting straps 120, e.g., across slot 116, to assist with holding brace 110 on user 10. As an example, mounting straps 120 may be hook-and-loop mounting straps. Thus, mounting straps 120 may include hook-and-loop features for selectively cinching mounting straps 120. As another example, mounting straps 120 may include buckles, bungee cords, buttons, etc. for selectively cinching mounting straps 120. Grip support orthosis 100 may include a suitable number of mounting straps 120. For instance, mounting straps 120 may include two (2), three (3), four (4) or more mounting straps 120.

Grip support orthosis 100 may also include a support bar 140 disposed within brace 110. As an example, support bar 140 may be positioned proximate a bottom side 119 of brace 110, e.g., and thus may be positioned opposite slot 116 on brace 110. Bottom side 119 of brace 110 may be positioned on an underside of forearm 18 of user 10 when brace 110 is worn by user 10. Conversely, top side 118 of brace 110 may be positioned on a topside of forearm 18 of user 10 when brace 110 is worn by user 10.

Support bar 140 may assist with limiting bending of wrist 16 of user 10. For instance, support bar 140 may extend between wrist end portion 112 and forearm end portion 114 of brace 110 within brace 110. Thus, e.g., a length of brace 110 may be about equal to a length of brace 110 between wrist end portion 112 and forearm end portion 114 of brace 100. In such a manner, support bar 140 may be securely mounted on forearm 18 of user 10 with brace 110. Support bar 140 may be bent 142 at wrist end portion 112 of brace 110 in order to accommodate wrist 16 of user 10 when brace 110 is worn by user 10. For example, wrist 16 of user 10 may be received within the bend 142 of support bar 140 at wrist end portion 112 of brace 110. Support bar 140 may be constructed of a suitable rigid material, such as aluminum, plastic, etc. Support bar 140 may be constructed of a more rigid material than brace 110 such that support bar 140 assists with supporting brace 110 and resists bending.

As may be seen from the above, brace 110 may be securely attached to user 10 with mounting straps 120. Thus, brace 110 of grip support orthotic 100 may provide a structure for securing other portions of grip support orthotic 100 to user 10. As noted above, grip support orthotic 100 includes features for assisting user 10 with gripping an object. In particular, elongated strap 130 may assist user 10 with gripping an object, as discussed in greater detail below.

Elongated strap 130 may extend between a first end portion 132 and a second end portion 134. First end portion 132 of elongated strap 130 may be fixed to brace 110, e.g., at wrist end portion 112 of brace 110. For instance, first end portion 132 of elongated strap 130 may be sewn, riveted, etc. to brace 110. Second end portion 134 of elongated strap 130 may be selectively mountable to brace 110. For instance, second end portion 134 of elongated strap 130 may be mounted to brace 110 with hook and loop fasteners, snap fasteners, buttons, buckles, clips, etc. In certain example embodiments, second end portion 134 of elongated strap 130 includes hooks and/or loops of a hook-and-loop fastener interface and corresponding hooks and/or loops of the hook-and-loop fastener interface may be disposed on brace 110. The hooks and/or loops of the hook-and-loop fastener interface on second end portion 134 of elongated strap 130 may engage the corresponding hooks and/or loops of the hook-and-loop fastener interface on brace 110 to secure second end portion 134 of elongated strap 130 to brace 110.

Elongated strap 130 may have a length between first and second end portions 132, 134 of elongated strap 130. The length of elongated strap 130 may be advantageously selected to allow elongated strap 130 to wrapped onto and/or around an object that a user desires to grasp. For instance, the length of elongated strap 130 may be no less than ten inches (10″). In particular, the length of elongated strap 130 may be no less than twelve inches (12″) and/or no greater than thirty-six inches (36″). As another example, the length of elongated strap 130 may be no less than eighteen inches (18″) and/or no greater than thirty inches (30″). Elongated strap 130 may be constructed of or with any suitable material. For instance, elongated strap 130 may be constructed of or with fabric webbing, rope, cord, leather, etc.

The above described sizing of the length of elongated strap 130 may advantageously allow elongated strap 130 to wrapped onto and/or around an object that a user desires to grasp. In addition, the above described sizing of the length of elongated strap 130 may advantageously allow elongated strap 130 to wrapped onto and/or around brace 110, e.g., to assist with securely mounting second end portion 134 of elongated strap 130 to brace 110. In such a manner, elongated strap 130 may assist user 10 with gripping the object. For example, user 10 may grasp the object, and then wrap elongated strap 130 onto and/or around the object. User 10 may then secure second end portion 134 of elongated strap 130 to brace 110 in order to prevent or limit unwinding of elongated strap 130 from the object. With elongated strap 130 so positioned, a grip of user 10 on the object is supplemented by elongated strap 130.

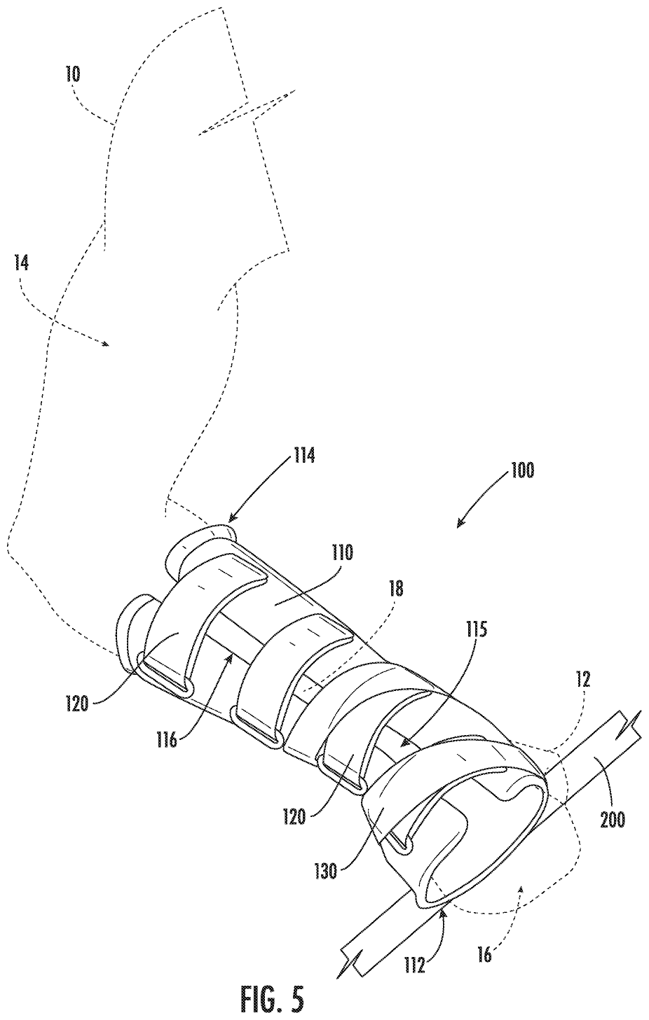

Grip support orthotic 100 with elongated strap 130 may be particularly useful when hand 12 and/or arm 14 of user 10 is, e.g., partially, paralyzed. Elongated strap 10 may be wound onto and/or around any suitable object that user 10 desires to grasp, such as a weight bar, a tool, etc. Thus, grip support orthotic 100 may assist user 10 by facilitating the grip of user 10 on the object. As a particular example, FIG. 5 is another perspective view of grip support orthotic 100 with elongated strap 130 wound onto a bar 200. Elongated strap 130 assist the grip of user 10 on bar 200, e.g., during weightlifting, yard work, etc.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. A grip support orthosis, comprising:
a brace having a wrist end portion and a forearm end portion, the brace configured to be worn by a user such that a wrist of the user is positioned in the brace at the wrist end portion of the brace and a forearm of the user is positioned in the brace at the forearm end portion of the brace;
a plurality of mounting straps on the brace, the plurality of mounting straps configured for selectively securing the brace on the user;
an elongated strap extending between a first end portion and a second end portion, the first end portion of the elongated strap fixed to the brace at the wrist end portion of the brace, the second end portion of the elongated strap selectively mountable to the brace, the elongated strap having a length between the first and second end portions of the elongated strap, the length of the elongated strap being no less than ten inches,
wherein the elongated strap is wrappable onto an object between the first and second end portions of the elongated strap in order to assist the user with gripping the object.

2. The grip support orthosis of claim 1, wherein the length of the elongated strap being no less than twelve inches and no greater than thirty-six inches.

3. The grip support orthosis of claim 1, wherein the plurality of mounting straps comprises a plurality of hook-and-loop mounting straps.

4. The grip support orthosis of claim 1, wherein the plurality of mounting straps comprises no less than three mounting straps.

5. The grip support orthosis of claim 1, further comprising a support bar disposed within the brace, the support bar positioned proximate a bottom side of the brace.

6. The grip support orthosis of claim 5, wherein the support bar extends between the wrist end portion and the forearm end portion of the brace within the brace.

7. The grip support orthosis of claim 6, wherein the support bar is bent at the wrist end portion of the brace in order to accommodate the wrist of the user when the brace is worn by the user.

8. The grip support orthosis of claim 5, wherein the support bar is an aluminum support bar.

9. The grip support orthosis of claim 5, wherein the brace defines a slot at a top side of the brace, the slot extending between the wrist end portion and the forearm end portion of the brace, the plurality of mounting straps extending across the slot.

10. The grip support orthosis of claim 9, wherein the bottom side of the brace is positioned on an underside of the forearm of the user when the brace is worn by the user, and the top side of the brace is positioned on a topside of the forearm of the user when the brace is worn by the user.

11. The grip support orthosis of claim 1, wherein the brace defines an opening at the wrist end portion of the brace, the opening configured for receipt of a thumb of the user.

12. The grip support orthosis of claim 1, wherein the second end portion of the elongated strap comprises one or both of hooks and loops of a hook-and-loop fastener interface for selectively mounting the second end portion of the elongated strap to the brace.

13. The grip support orthosis of claim 1, wherein the brace is a leather brace.

14. A grip support orthosis, comprising:
a brace having a wrist end portion and a forearm end portion, the brace configured to be worn by a user such that a wrist of the user is positioned in the brace at the wrist end portion of the brace and a forearm of the user is positioned in the brace at the forearm end portion of the brace, the brace defines a slot at a top side of the brace, the slot extending between the wrist end portion and the forearm end portion of the brace;
a plurality of mounting straps positioned on the brace proximate the slot, the plurality of mounting straps configured for selectively securing the brace on the user, the plurality of mounting straps extendable across the slot;
a support bar disposed within the brace, the support bar positioned proximate a bottom side of the brace;
an elongated strap extending between a first end portion and a second end portion, the first end portion of the elongated strap fixed to the brace, the second end portion of the elongated strap selectively mountable to the brace, the elongated strap having a length between the first and second end portions of the elongated strap, the length of the elongated strap being no less than ten inches,
wherein the elongated strap is wrappable onto an object between the first and second end portions of the elongated strap in order to assist the user with gripping the object.

15. The grip support orthosis of claim 14, wherein the length of the elongated strap being no less than twelve inches and no greater than thirty-six inches.

16. The grip support orthosis of claim 14, wherein the support bar extends between the wrist end portion and the forearm end portion of the brace within the brace.

17. The grip support orthosis of claim 16, wherein the support bar is bent at the wrist end portion of the brace in order to accommodate the wrist of the user when the brace is worn by the user.

18. The grip support orthosis of claim 14, wherein the bottom side of the brace is positioned on an underside of the forearm of the user when the brace is worn by the user, and the top side of the brace is positioned on a topside of the forearm of the user when the brace is worn by the user.

19. The grip support orthosis of claim 14, wherein the brace defines an opening at the wrist end portion of the brace, the opening configured for receipt of a thumb of the user.

20. The grip support orthosis of claim 14, wherein the second end portion of the elongated strap comprises one or both of hooks and loops of a hook-and-loop fastener interface for selectively mounting the second end portion of the elongated strap to the brace.
